(19) United States
(12) Patent Application Publication
(10) Pub. No.: US 2007/0015981 A1
(43) Pub. Date: Jan. 18, 2007

(54) Device and methods for the detection of locally-weighted tissue ischemia

(76) Inventors: David A. Benaron, Portola Valley, CA (US); Ilian H. Parachikov, Belmont, CA (US)

Correspondence Address: DORSEY & WHITNEY LLP, 555 CALIFORNIA STREET, SUITE 1000, SUITE 1000, SAN FRANCISCO, CA, 94104, US

(21) Appl. No.: 11/451,681

(22) Filed: Jun. 12, 2006

Related U.S. Application Data
(63) Continuation-in-part of application No. 10/651,541, filed on Aug. 29, 2003, now Pat. No. 7,062,306.

(51) Int. Cl. A61B 5/00

(52) U.S. Cl. 600/323; 600/332; 600/334

(57) ABSTRACT

An implantable ischemia detection device in which a broadband light source produces a continuous, visible, broadband light illuminating capillary sites within a well localized target tissue is provided. Light backscattered by the target tissue is collected by a sensor, allowing for an index of ischemia to be determined, and subsequently transmitted by a sending unit. Optionally, the device may be provided with an internal power source, the entire device encapsulated by a biocompatible shell to add long-term safety while implanted at a target tissue site.

### CROSS-REFERENCE TO RELATED APPLICATIONS

This application is a continuation-in-part of U.S. patent application Ser. No. 10/651,541, filed on Aug. 29, 2003, the entire disclosure of which is incorporated herein by reference.

### FIELD OF THE INVENTION

The present invention relates to a device and methods for providing highly-localized measurements of tissue ischemia. More particularly, in some embodiments the present invention relates to a device comprising a visible light source, a sensor, a power source, and a transmitter embedded into a long-term implantable shell for the purpose of performing a real-time spectroscopic analysis of an in-vivo tissue perfusion that is sensitive to local tissue ischemia and insensitive to regional arterial and venous oxygenation.

### BACKGROUND INFORMATION

The clinical detection of ischemia—an insufficient delivery of oxygen to meet a tissue's metabolic needs—is unreliable. Ischemia is especially difficult to detect when the ischemia is due to a localized reduction of blood flow—such as during a heart attack or a stroke.

Existing laboratory tests for ischemia, such as serum enzyme-leakage tests, e.g., tests for cardiac isoenzymes after a heart attack, or EKG electrical tests, are insensitive indicators of such localized tissue ischemia, especially during its early stages. In such cases, any signal emanating from the locally ischemic tissue is obscured by signals coming from healthy tissues. Blood tests are also insensitive to local ischemia, since blood tests are not able to reveal the localized nature of the ischemia.

Ischemia does not result from the oxygenation of the arterial or venous blood when measured by a blood test in the large central arteries and veins, nor in the nearby capillaries in tissue that is not ischemia. Rather, ischemia is a result of low oxygenation in a local tissue as reflected in the oxygenation of a locally depressed capillary.

Non-invasive imaging of ischemia also lacks the immediacy that allows for early intervention or real-time feedback to other devices such as pacemakers.

Sensors designed to detect ischemia are rare in the art, and none of them detect tissue ischemia directly. For example, U.S. patent application Ser. No. 6,532,381 teaches the detection of ischemia using externally measured electrical (EKG) monitoring and microprocessor control. In U.S. Pat. No. 5,135,004, U.S. Publication No. 2004/0122478, and International Publication No. WO 00/64534, the presence of ischemia is predicted based upon an implantable device that measures the electrical (EKG), blood pressure, local pH, and/or physical (acceleration during contraction) characteristics of the heart. U.S. Pat. No. 6,527,729 discloses an implantable acoustic sensor that responds to heart failure by changes in the sound of the heartbeat. U.S. Pat. No. 5,199,428 and U.S. Publication No. 2004/0220460 teach implantable devices to monitor blood oxygenation (venous blood and arterial blood, respectively), in the latter case specifically rejecting local tissue saturation from encapsulation, thus teaching away from direct tissue monitoring. For reasons to be outlined in more detail later, such non-tissue blood oxygenation (whether arterial or venous) is insensitive to tissue ischemia, and is at best an indirect measure of tissue ischemia.

For each of the devices above, ischemia is measured only by indirect and unreliable indicators of ischemia, such as by indicators of cardiac electrical, mechanical, and acoustic dysfunction. Another point to consider is that organs other than the heart are common sites of ischemia (such as in the kidney, liver, or gut), and the prior art is not directed to these other organs at all, which may not contract or make sounds. Therefore, none of the above devices detect local tissue ischemia directly, nor can they be applied generally to any organ without regard to site.

All of the above devices are limited by being either indirect measures of local ischemia, or by being insensitive to local ischemia. None of the prior devices or methods allow for a direct highly local-weighted detection of ischemia in a broad array of target sites. Such a system has not been previously described, nor successfully commercialized.

In view of the foregoing, there is a need in this art for a device and methods for detecting local ischemia in a tissue without regard to site.

There is a further need in this art for a device and methods for performing a real-time spectroscopic analysis of an in-vivo tissue perfusion that is sensitive to local tissue ischemia and insensitive to regional arterial and venous oxygenation.

There is also a need in this art for a device and methods for providing a quantitative measure or index of localized ischemia.

### SUMMARY OF THE INVENTION

In view of the foregoing, a general aspect of the present invention is to provide a device and methods for detecting local ischemia in a tissue without regard to site. The present invention teaches that the site at which ischemia occurs is always local, and that local tissue in nearly every case will attempt to compensate for local ischemia, producing a direct effect of early ischemia upon capillary hemoglobin saturation, well in advance of acidosis and metabolic failure, and even when local flow may, in fact, be increased. This local early effect is most often not measurable using standard monitoring of arterial and/or venous blood, or by global measures including central venous oxygenation and cardiac output, among others. Further, capitalizing upon the sensitivity and reliability of this local capillary effect allows for the design of a highly-localized ischemia detector.

In one aspect of the present invention, there is provided a device for detecting highly-localized tissue ischemia in a target tissue. The highly-localized ischemia detector comprises a power source, a broadband light source, a sensor, and a sending unit, which may be a transmitter for sending signals sensed by the sensor to external processing devices, such as an external ischemia monitor. In one exemplary embodiment, the broadband light source comprises a phosphor-coated white LED to produce continuous, broadband, visible light from 400 nm to 700 nm, which is transmitted directly to the target tissue. Scattered light returning from the target tissue is detected by a wavelength-sensitive detector, and a signal highly related to local ischemia is generated using this wavelength-sensitive information via spectroscopic analysis.

Advantageously, the highly-localized ischemia detector of the present invention may detect ischemia using light, which allows for simple, safe, and non-electrical transmission of the measuring photons as required. Another advantage is that the highly-localized ischemia detector according to some embodiment of the present invention enables a physician or surgeon to obtain real-time feedback regarding local tissue ischemia in high-risk patients, and to respond accordingly, while any injury remains reversible. A further advantage is that the device of the present invention may be safely deployed within a living body to give long-term tissue-specific feedback as needed.

In some embodiments a highly-localized ischemia detector of the present invention may be actively coupled to a therapeutic device, such as a pacemaker, to provide feedback to the pacing function, or passively coupled to a therapeutic device, such as applied to a stent to monitor stent performance over time, based upon the detection and degree of local ischemia. Ischemia sensing may be used to enable detection of many types of disease, such as tissue rejection, tissue infection, vessel leakage, vessel occlusion, and the like, many of which produce ischemia as an aspect of the disease.

Embodiments of the present invention further provide a device for detecting local ischemia in a tissue, characterized in that the device is configured such that wavelengths of light are selectively emitted, and the selective wavelengths are substantially transmitted through capillaries in tissue while being substantially absorbed by arterial and venous vessels in the tissue.

### BRIEF DESCRIPTION OF THE DRAWINGS

The breadth of uses and advantages of the present invention are best understood by example, and by a detailed explanation of the workings of a constructed device. These and other advantages of the present invention will become apparent upon consideration of the following detailed description, taken in conjunction with the accompanying drawings, in which like reference characters refer to like parts throughout, and in which:

FIG. 1 is an exemplary schematic diagram of an implantable ischemia detector constructed in accordance with the principles and embodiments of the present invention;

FIGS. 2A-F are exemplary schematic diagrams showing various configurations of the wavelength-resolved light sensor of FIG. 1 and constructed in accordance with principles and embodiments of the present invention;

FIGS. 3A-B are exemplary graphs illustrating how differently vascular hemoglobin may be sampled using visible and infrared light;

FIG. 4 is an exemplary graph showing calculated estimates of the proportion of a signal detected by the highly-localized ischemia detector of FIG. 1 and coming from the vessels (conventional sites of measurement in the arteries and veins) versus the proportion of the signal coming from the capillaries across a range of wavelengths from ultraviolet to infrared;

FIG. 5 is an exemplary schematic diagram of a medical monitor system incorporating the principles and embodiments of the present invention;

FIG. 6 is an exemplary graph showing the relationship between blood flow and colon ischemia in an animal subject under controlled heart output; and

FIG. 7 is a flow diagram of an exemplary embodiment of local ischemia detection performed by a ischemia detection device constructed in accordance with embodiments of the present invention.

### DETAILED DESCRIPTION OF EMBODIMENTS OF THE INVENTION

Generally, the present invention provides devices and methods for detecting local ischemia in a target tissue without regard to tissue site and for producing a target signal generating a direct, quantitative measure or index of ischemia. Tissue, as used herein, may be any material from a living animal, plant, viral, or bacterial subject, with an emphasis on mammals, especially humans. A target tissue may be a tissue material to be detected, imaged, or studied. A target signal may be a detected signal specific to the desired target tissue. This signal may be enhanced through use of known optical techniques, including use of a contrast agent, scattering, absorbance, phosphorescence, fluorescence, Raman effects, or other known spectroscopy and signal processing techniques, provided only that such techniques are applied in a manner to perform substantially locally and capillary weighted ischemia sensing.

Ischemia, as used herein, may be a condition in which the perfusion of a tissue, i.e., the delivery or flow of blood and oxygen to a tissue, is locally inadequate to meet its metabolic needs. Ischemia is distinguished from flow in that low flow alone does not guarantee ischemia (such as during tissue cooling), nor does high flow rule out ischemia (such as during sepsis or when the blood delivered does not contain adequate oxygen). Ischemia is a co-existing condition in many different types of illnesses, including infection (sepsis), tissue rejection (host vs. graft disease), heart attack (myocardial ischemia), stroke (cerebral ischemia), acute or chronic organ failure, diabetic peripheral vascular disease, and other conditions.

In accordance with the present invention, the device and methods for detecting local ischemia in a target tissue site are based on the use of a light source. As used herein, a light source is a source of illuminating photons. It may be composed of a simple light bulb, a laser, a flash lamp, an LED, a white LED, or another light source or combination of sources, or it may be a complex form including a light emitter such as a bulb or LED, one or more filter elements, a transmission element such as an integrated optical fiber, a guidance element such as a reflective prism or internal lens, and other elements intended to enhance the optical coupling of the light from the source to the tissue or sample under study. The light may be generated using electrical input (such as with an LED), optical input (such as a fluorescent dye in a fiber responding to light), or any other source of energy, internal or external to the source. The light source may be continuously on, pulsed, or even analyzed as time-, frequency-, or spatially-resolved. The light emitter may comprise a single or multiple light emitting elements, such as a combination of different light emitting diodes to produce a spectrum of light.

In the preferred embodiment, the light source used in the device of the present invention comprises a broadband light source emitting broadband light, which is light produced over a wide range of wavelengths sufficient to perform solution of multiple simultaneous spectroscopic equations. For a tissue site, a wavelength width of at least 40 nm is likely to be needed. In one exemplary embodiment, a broadband white light-emitting diode (“LED”) is used. A broadband white LED produces light from 400 nm to beyond 700 nm and is often comprised of a blue LED and a blue-absorbing broad-emitting phosphor that emits light over a wide range of visible wavelengths. Visible wavelengths include electromagnetic radiation from blue to yellow, namely between 400 nm and 625 microns, but especially those from green to orange, i.e., between 475 and 600 nm, where the absorbance by capillary hemoglobin and/or cytochrome pigments is the strongest. As used in the examples herein, other phosphors and any broadband LED could be used, even if not emitting over a full (white) spectrum. For example, a green LED emitting over a FWHM range of 100 nm would also be considered to be broadband without deviating from the principles of the present invention.

Aspects of the present invention further provide for the transmission of the broadband light to a target tissue site, absorbance of the light by the tissue site, and reflectance or scattering of the light back into a light detector for generating a measurable signal in response to the light incident on the detector. As used herein, absorbance of light by a tissue site refers to the fractional transmission of light absorbed by the tissue. For example, if the initial intensity of light reaching a tissue is Io, and the light passing through the tissue is I, then the transmittance T of light is defined as T=I/Io. Putting numbers in, if the light level in arbitrary units reaching a tissue is 2, and the light passing through a tissue is 1, then the effect transmittance T is (½)=0.5. This value is related to effective absorbance (to a first approximation in light scattering tissue) as absorbance A=log (1/T). Thus a 10% transmittance corresponds to an absorbance A=1, while a 1% transmittance corresponds to an absorbance A=2.

Embodiments of the present invention provide devices and methods for tuning a light source to selectively emit one or more wavelengths, the one or more wavelengths being selected such that the light is substantially transmitted through capillaries present in the tissue, while being absorbed by arterial and venous vessels in the tissue, thus providing a locally-weighted measure of ischemia.

In the accompanying examples, one target tissue site is the intestinal mucosa, which is the thin lining covering the inner surface of the gut. Deeply penetrating infrared wavelengths would have difficulty focusing upon such a thin target tissue, and would likely report instead tissues far away from the mucosa, such as supporting structures in the abdominal cavity. This can lower sensitivity greatly, as the high-metabolism tissue is the gut mucosa, which turns over quickly (shed and replaced every 5-7 days), and thus has a high oxygen need. The mucosa is the tissue which shows ischemia most readily within the gut, and by measuring the mucosa, supporting structures, abdominal muscle, and other tissues, the biggest drop in tissue hemoglobin saturation that occurs in the mucosa would be diluted out by other stable tissues unless the wavelength selection is done to allow local and capillary monitoring only.

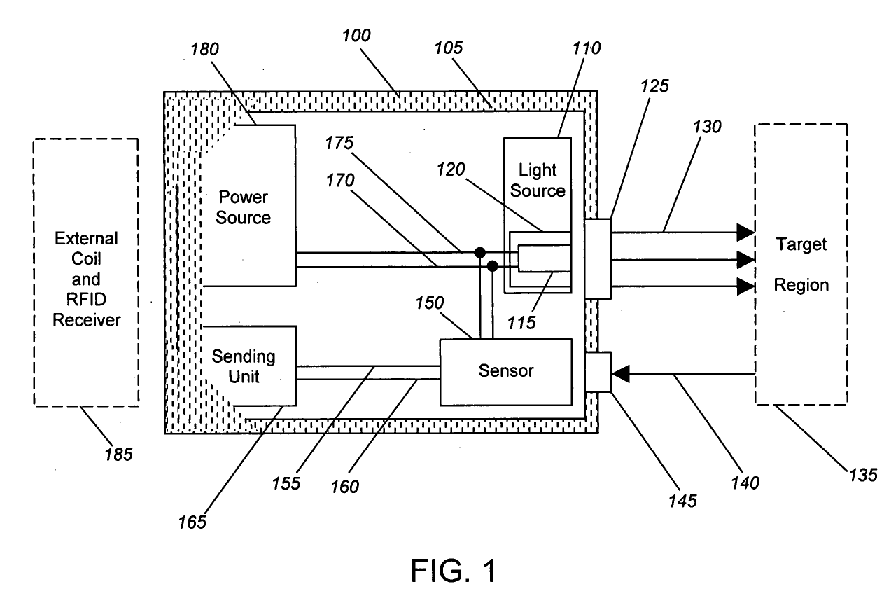

Referring now to FIG. 1, an exemplary schematic diagram of an implantable ischemia detector capable highly-localized ischemia detection and constructed in accordance with principles and embodiments of the present invention is provided.

Ischemia detection device 100 is surrounded by biocompatible exterior 105. Typically, exterior 105 is constructed from approved Class VI materials as recognized by the United States Food and Drug Administration (“FDA”) or other medical device regulatory agencies, such as polyethylene or surgical steel. Portions of the sensor, power supply, light source, or transmitter in device 100 may protrude as needed from this shell within the spirit of this invention, provided that the protruding parts themselves are biocompatible.

Within device 100, light source 110 is illustrated in its component parts. In one exemplary embodiment, broad spectrum white light is emitted by a high conversion-efficiency white LED source (in this case, The LED Light, model T1-3/4-20W-a, sold by Fallon, N V). LED source 115 is embedded into a plastic beam-shaping mount using optical clear epoxy 120 to allow light generated in LED 115 to be collimated, thus remaining at a near-constant diameter after passing through optical window 125 to leave device 100. Light then is able to pass forward as shown by light path vectors 130, with at least a portion of this light optically coupled to target region 135. Note that while target region 135 may be in some instances a living tissue, the tissue itself is not considered to be a claimed part of this invention.

A portion of the light reaching target 135 is backscattered and returns to device 100, as shown by light path vectors 140, to optical collection window 145. Collection window 145 in this embodiment is a glass, plastic, or quartz window, but can alternatively be merely an aperture, or even be a lens and the like, as appropriate. Light then strikes sensor 150, where it is sensed and detected.

Sensor 150 may be comprised of a number of discrete detectors configured to be wavelength-sensitive, or may be a continuous CCD spectrometer, with entry of light by wavelength controlled gratings, filters, or wavelength-specific optical fibers. In any event, sensor 150 transmits an ischemia signal related to the detected light backscattered from target 135, producing an electrical signal sent via wires 155 and 160 to transmitter chip or sending unit 165.

Light source 110 also has two electrical connections 170 and 175, connecting light source 110 to power source 180. In this embodiment, power source 180 is an inductive power supply, capable of receiving an inductive field from externally powered coil and RFID receiver 185 placed outside of the body, in order to produce power for device 100 as required. Note that external powered coil 185 is shown for purposes of example and illustration only, and is not considered a required part of the present invention. Alternatively, power source 180 could merely be a long-lived implantable battery, in which case an external powered coil may not be required at all.

Operation of device 100 may now be described. Device 100 may be implanted in a patient, for example, in the chest wall of a patient undergoing coronary artery repair for heart disease. The patient is allowed to heal after surgery, and implantable device 100 may be left inside the patient's body, without a direct physical connection to the outside world.

Device 100 may measure ischemia at the muscle directly, or it can be placed at a distance. In the latter case, vectors 130 are fiber optics extended from device 100 and into close proximity to the target heart muscle, sufficient for optical coupling. As used herein, optical coupling refers to the arrangement of two elements such that light exiting the first element interacts, at least in part, with the second element. This may be free-space (unaided) transmission through air or space, or may require use of intervening optical elements such as lenses, filters, fused fiber expanders, collimators, concentrators, collectors, optical fibers, prisms, mirrors, or mirrored surfaces.

In this example, device 100 is normally powered down and in a resting (off) state. At some point, it is desired to test the target heart muscle for the presence of ischemia. Through inductive coupling, external coil 185 induces a current in inductive power source 180 located within device 100, producing sufficient power for device 100 to power up and turn on. Light source 110 begins to illuminate target 135, in this case, the patient's heart muscle. Sensor 150, which may be an embedded spectrophotometer, receives backscattered light, and resolves the incoming light by wavelength, a marker of ischemia.

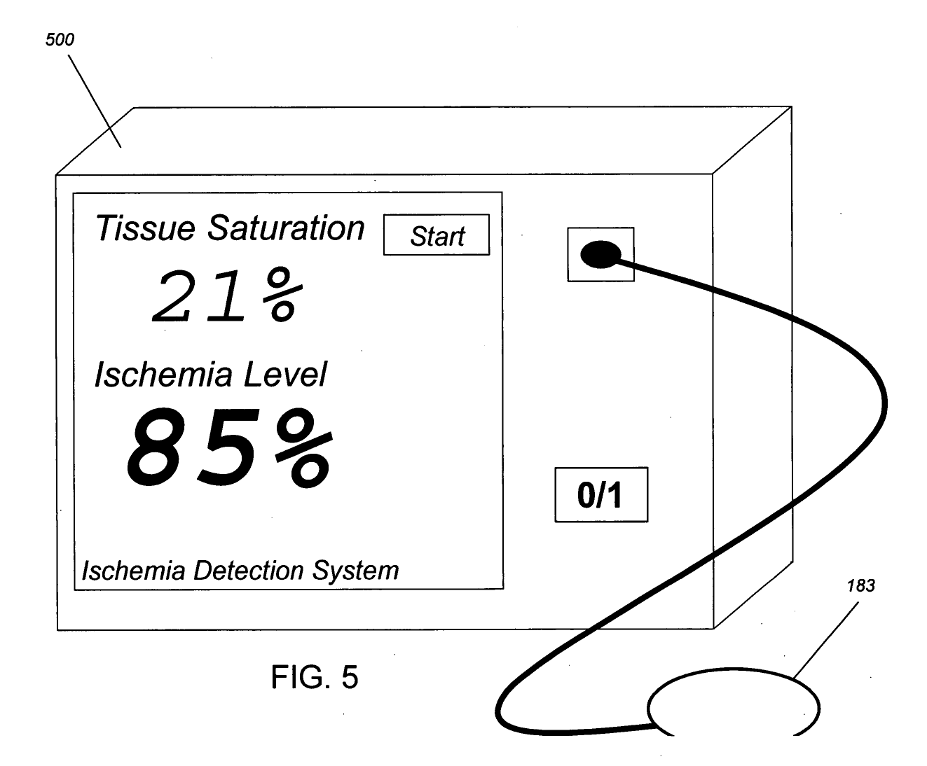

The result of this determination is sent to sending unit 165, which may be an RF transmitter that sends the sensed signals to external coil 185, which may also contain an RFID receiver. There, the signal received by external coil 185 may be processed for the oxygenation of the hemoglobin in the terminal capillary beds, a marker of ischemia, by external monitor 500 of FIG. 5, as shown in the data described herein below.

Once the measurement is completed, external coil 185 is moved away from device 100, and device 100 powers down and returns to a resting state.

In an alternative embodiment, power source 180 may be charged during proximity to external coil 185, or have an internal battery source, allowing device 100 to operate when external coil 185 is not present. Sending unit 165 may then transmit without being directly queried, such as in response to a dangerous level of ischemia.

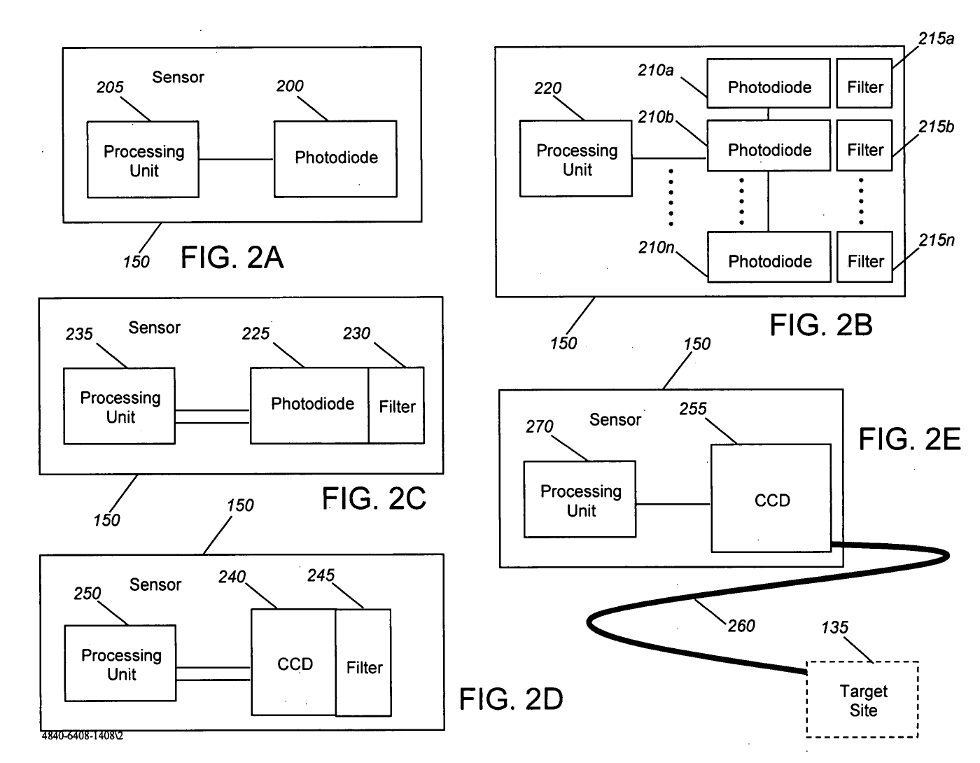

Referring now to FIGS. 2A-F, exemplary schematic diagrams showing various configurations of the wavelength-resolved light sensor of FIG. 1 and constructed in accordance with the principles and embodiments of the present invention are provided. In one configuration, shown in FIG. 2A, sensor 150 comprises single photodiode 200 and processing electronics unit 205. Photodiode 200 is made wavelength sensitive through the design of LED 110 as a cluster of LEDs of different wavelengths, each emitting at a different time or modulation frequency to allow decoding of the illuminating wavelength by photodiode 200 and processing electronics unit 205. Alternatively, as shown in FIG. 2B, sensor 150 may comprise a set of different photodiodes 210a-n, each with filters 215a-n, allowing each photodiode to be sensitive to only one wavelength range, again allowing decoding of the sensed light by wavelength by processing electronics unit 220. Alternatively again, as shown in FIG. 2C, sensor 150 may be single photodiode 225 with electronically variable filter 230, allowing the wavelength transmitted to be selected and processed by processing electronics unit 235.

In yet another configuration, as shown in FIG. 2D, sensor 150 may be CCD chip 240 with filter window 245 that varies over its length, allowing only certain wavelengths to reach each portion of CCD 240, allowing decoding of the illuminating wavelength by processing electronics unit 250. Finally, in the preferred embodiment shown in FIG. 2E, sensor 150 comprises CCD chip 255 with optical fibers 260 attached to CCD 255 in a linear array. Fibers 260 are manufactured such that each fiber has a different interference coating on end 265 (not shown), allowing each fiber to transmit a different narrow wavelength range, allowing decoding of the illuminating wavelength by processing electronics unit 270. Fibers 260 are biocompatible and can extend outside of device case 115, allowing device 100 to be placed remotely at the target to be monitored, and for the free end of fibers 260 to be placed in proximity to target site 135.

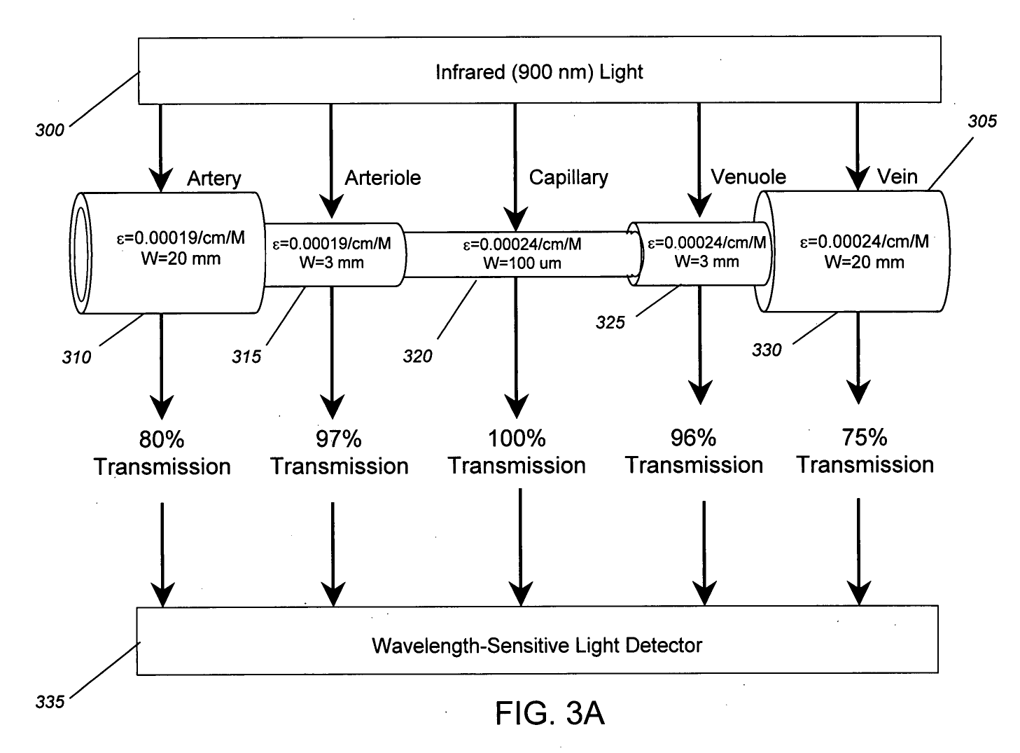

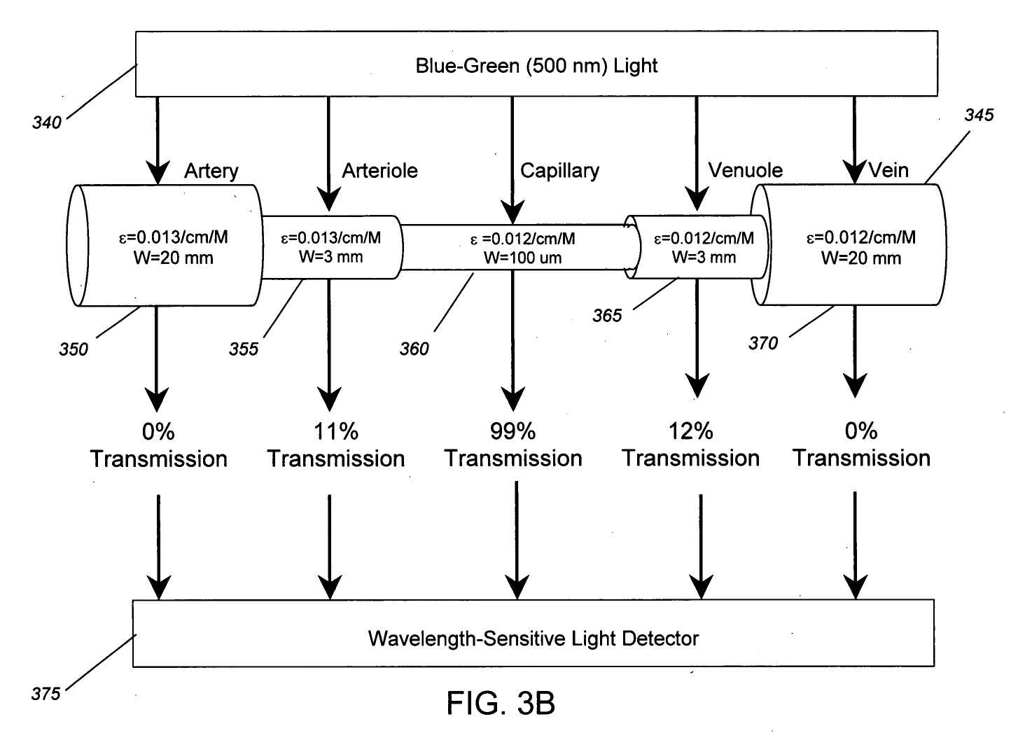

The selection of wavelengths is best understood by an example of how different wavelengths are absorbed by arterial, venous, and capillary vessels. Referring now to FIGS. 3A-B, exemplary graphs illustrating how differently vascular hemoglobin may be sampled using visible and infrared light are provided. In the first case, shown in FIG. 3A, light 300 consists of infrared light (900 nm) shined onto vascular system 305. Vascular system 305 is composed of a series of blood vessels and branches, through which blood flows, as illustrated in a linear schematic showing artery 310, arteriole 315, capillary 320, venuole 325, and vein 330. In the living body, blood red with oxygen, would flow from artery 310 into arteriole 315, then into capillary 320 where oxygen exchange with the local tissue occurs according to the tissue's needs. Blood then continues on through venuole 325, and finally into vein 330 prior to leaving the region.

It is a critical aspect of tissue physiology that each part of vascular system 305 has a characteristic width (W) and total absorbance (A), as well as a unique physiological function. As given by a first approximation, the total light absorbance A of light in a structure is estimated by the following equation:
A=ε×W×C (1)
where ε is the specific absorbance or light extinction coefficient, that is, the absorbance per unit distance of hemoglobin at a particular wavelength, W is the width of the vessel, and C is the concentration of the hemoglobin.

As the body's blood concentration is somewhat constant across all of the vessels, the relative difference in absorbance of light comes from the varying width and prevalence of the vessels in the tissue. Because the total absorbance A is logarithmically related to the intensity of the transmitted light, a one unit increase in absorbance (say, from 1 to 2) results in a ten-fold decrease in the transmission of light. This is critical to the detection of local ischemia, as the scaling of absorbance with vessel width results in one-thousand-fold reduced transmittance of light by large vessels compared to small vessels at certain wavelengths, while having similar transmittance between large and small vessels at other wavelengths. The inventors have discovered that the choice of wavelength allows one to tune the weighting of the detected signal toward or away from the capillary bed, if desired.

This absorbance across blood vessels is illustrated for infrared light in FIG. 3A. As shown in FIG. 3A, artery 310 has a typical arterial 99% oxygenated hemoglobin, with 1% deoxygenated hemoglobin, so that its absorbance more closely approximates a solution of pure oxyhemoglobin, with a light extinction coefficient e of 0.0019/cm/M. Also, arteries in general are large vessels, such that artery 310 has a width W of 20 mm. Note that this width is for illustration purposes only, as some arteries such as the aorta can be as wide as 50 mm or more, while others can be only 5 mm across. Because the absorbance of light in the infrared region is low and the transmittance is high, even with a 20 mm width nearly 80% of the light crossing artery 310 reaches detector 335.

After this blood leaves artery 310, it enters arteriole 315. In general, arterioles are smaller than arteries, but larger than capillaries. Because the blood in arteriole 315 is still well-oxygenated arterial blood, the extinction coefficient e of arteriole 315 is the same as the extinction coefficient e of artery 310. However, because arteriole 315 is only 3 mm wide rather than 20 mm wide, 97% of the light entering arteriole 315 emerges out the other side to be detected by detector 335.

Next, this blood passes into tissue capillary 320, where the blood is in intimate contact with the tissue. This is critical, as oxygen leaves capillary 320 and the blood increases its concentration of de-oxygenated hemoglobin. Deoxyhemoglobin has a larger extinction coefficient e at this wavelength than does oxygenated hemoglobin. However, this is balanced by the fact that capillary 320 is so thin, perhaps 10-100 microns wide, that nearly 100% of the light passing through capillary 320 reaches detector 335.

Once this blood leaves capillary 320, it flows into venuole 325 and then into vein 330. Because the absorbance of deoxygenated hemoglobin is higher than for the same volume of oxygenated hemoglobin, there is a reduced transmittance of light through these vessels to detector 335. Namely 96% and 75% of the light reaching venuole 325 and vein 330, respectively, survive to be detected by detector 335.

There is one additional factor to consider. There are more capillaries by cross sectional volume than there are arteries in living tissue, which overall is about 8% of the body by blood vessels by volume in tissue. When one takes into account the relative number of these vessels (at a deeply penetrating infrared light depth of up to 6 cm, the fractional cross-sectional tissue volumes are 0.5% arteries, 1% arterioles, 5% capillaries, 1% venuoles, and 0.5% veins, for a total of 8% blood vessels by volume in tissue), it can be determined that of the light that has crossed a blood vessel, 65% of that light reaching detector 335 has passed through the capillaries such as capillary 320.

This gives a heavy weighting to larger vessels (35%), and can generate inaccuracies and insensitivity to ischemia, as shall be described herein below in additional examples.

The inventors have discovered that the above situation is very different when considering light in a specific wavelength range, such as a range of 400 to 600 nm, and more specifically blue to green visible illuminating light (at around 500 nm), which penetrates larger vessels very poorly. This is a wavelength taught away from by oximetry art, which instead is focused on the advantages of near infrared light.

Use of 500 nm light for illumination of tissue is illustrated in FIG. 3B. There, light 340 in the 500 nm wavelength range is shined onto vascular system 345, consisting of artery 350, arteriole 355, capillary 360, venuole 365, and vein 370, analogous to vascular system 305 shown in FIG. 3A.

Each part of vascular system 340 has the same width (W) as their counterparts in vascular system 305 shown in FIG. 3B, but now with a one-hundred-fold higher extinction coefficient e due to the relatively increased absorbance of green to orange light by blood. Because photon loss over distance is a power function, this increase in effective absorbance has large effects. For example, if 90% of a certain wavelength of light is transmitted across a vessel per centimeter traveled, the light passing through a 3 cm vessel is about 72% (90% transmission for each cm of travel). If, on the other hand, the absorbance is now increased ten-fold, then just 10% of the light passes though a centimeter vessel, while 0.1% of the light transmits through a 3 cm vessel. A higher absorbance thus favors transmission preferentially through the smaller vessels, which then dominate the detected signal.

This effect of higher absorbance can now be seen in FIG. 3B by working through the same calculations as described above with reference to FIG. 3A. Because absorbance in the visible range is high, and the width of the artery is so large, virtually no light is transmitted from artery 350 or vein 370 to detector 375.

After blood leaves artery 350, the blood enters arteriole 355. Just before the blood enters vein 370 it is in venuole 365. Because the extinction coefficients are still high, only 11-12% of light crossing arteriole 355 or venuole 365 is transmitted to be detected by detector 375.

In contrast, the thin capillaries are still quite transparent, even at this high absorbance, with 99% of the light passing through capillary 360 reaching detector 375. This means that nearly all of the light at 500 nm that reaches detector 375 has come from the capillaries. Further, 500 nm light penetrates less deeply, such that the prevalence of larger vessels is reduced in the sampling region.

In the final analysis, whereas 35% of the signal comes from non-capillary vessels using infrared light, only 8% comes from non-capillary vessels at 500 nm, and only 2l% from non-capillary vessels at 400 nm. Thus, according to the present invention, by using a wavelength selected to be relatively transmitted by capillaries, and not by larger vessels, the sensitivity of the measurement to ischemia has been increased.

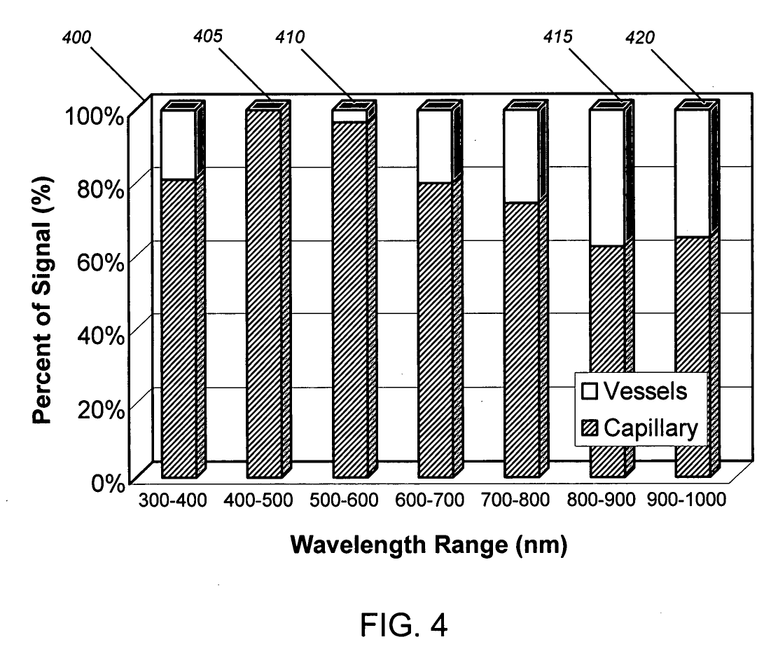

Referring now to FIG. 4, an exemplary graph showing calculated estimates of the proportion of a signal detected by the highly-localized ischemia detector of FIG. 1 and coming from the vessels (conventional sites of measurement in the arteries and veins) versus the proportion of the signal coming from the capillaries across a range of wavelengths from ultraviolet to infrared is provided. When analyzed across many wavelength regions, from ultraviolet to infrared, the percent of signal passing through the capillaries and reaching the light detector, such as light detector 150 of FIG. 1, can be seen to be maximized in the 400-600 nm range, as shown in columns 405 and 410 in graph 400. In contrast, the percent of signal passing through the larger vessels and reaching the light detector can be seen to be maximized in the 800-1000 nm range, as shown in columns 415 and 420 of graph 400.

It is this difference in transmittance of the wavelengths that enables one to make locally-weighted and microvascular-weighted measurements to detect ischemia in a local portion of a target tissue site. A locally-weighted measurement, as used herein, is a measurement that is weighted toward the condition of a local tissue near a sensor probe, rather than the blood flowing in the larger vessels that is not in physiological contact, e.g., capable of direct and significant oxygen exchange, with that local tissue. A microvascular-weighted measurement is a measurement that is weighted toward the smallest vessels, such as those having 20 microns or smaller, rather than to the blood flowing in the larger vessels that is not in physiologic contact with the local tissue.

Due to the deep penetration of large vessels by infrared (and red) light, using infrared or red light to measure light transmittance and absorbance through tissue reflects a wide range of vessel sizes and results in measurements that are not substantially locally-weighted or microvascularly-weighted. In contrast, a blue-green weighted measurement penetrates larger vessels poorly but capillaries well, and does not travel to sufficient depths that would force inclusion of many large vessels. That is, using blue-green light to measure light transmittance and absorbance through tissue results in a substantially locally-weighted and microvascular-weighted measurement. This is non-obvious and counterintuitive to the prior art, which tends to teach the use of infrared light for its tissue-penetrating ability and against the use of the shallow-penetrating blue end of the visible spectrum.

The importance of the right wavelengths for detecting a capillary-only signal as described above is critical for a proper detection of ischemia. The capillary bed is the primary site of oxygen exchange between tissues and the circulatory system, while measurement at other tissues will result in an insensitive and non-localized measurement.

Consider a patient with normal arterial oxygenation (such as 98%) and normal venous oxygenation (such as 65%). These data are shown below in Table 1, before and after two ischemic conditions, arrest and reduced flow.

TABLE 1
Effect of Wavelength on Measured Saturation
Under Normal and Two Ischemic Conditions

| | | | | | | | |
|---|---|---|---|---|---|---|---|
| | Capillary | Arterial | Venous | Capillary | Measured | Normal | Outside of |
| Wavelength | Fraction | Saturation | Saturation | Saturation | Saturation | Range | Normal? |

ARREST

| | | | | | | | |
|---|---|---|---|---|---|---|---|
| Visible (500 nm) | | | | | | | |
| Normal Baseline | 0.94 | 98% | 70% | 74% | 75% | [62-78] | No |
| After Arrest | 0.94 | 98% | 70% | 50% | 52% | [62-78] | Yes |
| Infrared (900 nm) | | | | | | | |
| Normal Baseline | 0.62 | 98% | 70% | 74% | 78% | [48-84] | No |
| After Arrest | 0.62 | 98% | 70% | 50% | 63% | [48-84] | No |

REDUCED FLOW

| | | | | | | | |
|---|---|---|---|---|---|---|---|
| Visible (500 nm) | | | | | | | |
| Normal Baseline | 0.94 | 98% | 70% | 74% | 75% | [62-78] | No |
| After Arrest | 0.94 | 98% | 55% | 59% | 60% | [62-78] | Yes |
| Infrared (900 nm) | | | | | | | |
| Normal Baseline | 0.62 | 98% | 70% | 74% | 78% | [48-84] | No |
| After Arrest | 0.62 | 98% | 55% | 59% | 66% | [48-84] | No |

For normal patients, saturation measured using visible or near-infrared light will often give similar results. For example, for normal patients measured at baseline in the 900 nm wavelength range (row 3), the signal is fractionally 62% from the capillaries (row 3, column 1) and has a saturation of 74% (row 3, column 4). The remaining 38% of the blood volume therefore comes from the larger vessels (i.e., 100-62=38) and has an average saturation (averaged from the arterial (row 3, column 2, 98%) and venous (row 3, column 3, 70%) saturation) of 84%, for a mean optical saturation of 78% (row 3, column 5).

In comparison, the signal measured at baseline in the 500 nm (visible) range (row 1) is fractionally 94% from the capillaries (row 1, column 1), with a final mean optical saturation of 75% (row 1, column 5). Under such normal circumstances, both near-infrared and visible approaches yield essentially the same value, i.e., 78% for near-infrared and 75% for visible, and both values are well within the normal range.

The situation, however, is very different during ischemic states.

First, consider what happens if the patient arrests. Because the blood within vessels has stopped, and is no longer flowing, the arterial blood stays oxygenated within the arteries, while the venous blood stays partially oxygenated just as it was when it left the capillary bed, for neither arterial nor venous blood remains in communication with the tissue. The capillary blood, which remains in the capillaries and supplying oxygen, drops quickly over time. In this example, it drops to 50% after a few seconds of ischemia (rows 2 and 4, column 4).

Now, from Table 1, let us again calculate the measured value for tissue saturation in this arrested patient. In the 900 nm range, the signal is still fractionally 62% from the capillaries, with an average saturation (mid-arterial-venous) of 84%, and fractionally 38% from the larger vessels, but now with a decreased capillary saturation of 50%. This yields a mean measure saturation of 63% (row 4, column 5). Because the published standard deviation of near-infrared approaches is ±9%, the normal range is defined as the mean ±2 SD, or 48%—84%. In this case, the near infrared value of 63% falls well within this normal near-infrared range.

In comparison, in the 500 nm range, the signal is fractionally only 6% from the larger vessels, with a saturation now decreased to 50%, and fractionally 94% from the capillaries with an arterial/venous average of 84% (98% arterial averaged with 70% venous) in this example. This yields a measured tissue saturation of 52% (row 2, column 5). For visible light methods, the standard deviation in vivo is ±4%, for a normal range of 62%-78%. Clearly, the visible light method shows this 52% to be over two standard deviations outside of the normal range.

This demonstrates that near-infrared methods are significantly less sensitive to ischemia than visible methods for fundamental optical and physical reasons.

It is important to note that the measurement of flow/perfusion alone, the measurement of blood oxygenation (not tissue oxygenation, but oxygenation of the arterial blood), the presence of lactate or acid pH (or pHi), and cardiac output alone are not sufficient to detect the condition of ischemia.

In cases of hypothermia (low body temperature), the oxygen consumption of the tissue is reduced by a factor of two for every ten degrees of cooling. Thus, if a patient has a blood flow of one times, say, X, at a normal temperature, the blood flow may be reduced to ¼ times X when the body temperature drops twenty degrees. Using only flow, a physician could conclude that the ¼ time X flow is far too low—in fact, ischemic—when the reduced flow in this case perfectly matches the body's needs at that reduced temperature. Similarly, during fever, the body's needs increase by the same rule, such that a normal flow at severely elevated body temperatures could be interpreted as fine, when in fact the normal flow is insufficient to meet the body's needs.

Similarly, a normal lactate level, a key measure of ischemia in patients, is not sufficient to rule out ischemia. For example, in one patient using the present invention, the flow to the colon was completely stopped. Because there was absolutely no blood flow, the tissue was by definition ischemic (unless metabolism was also driven to nearly zero, such as with cryopreservation). However, because no blood was leaving the tissue to reenter the circulation, the blood lactate levels and pH were normal. In this case, the present invention showed oxygen levels dropping to nearly zero from near-normal values, allowing a diagnosis of ischemia. When a surgery was performed to restore blood flow to the colon, the lactic acid within the tissue was washed into the blood circulation, and the patient developed high lactate levels and low pH, both diagnostic of ischemia, though developing them only once before the colon was repaired. Had the lactate levels and low pH been waited for before performing a revascularization, the colon would have died before blood supply was restored.

This raises the question: if not low flow, low pH, or low venous saturation, how can ischemia be detected? Going back to physiology, ischemia results when the tissues receive insufficient oxygen delivery. The manner in which the body senses this is by adjusting capillary hemoglobin saturation. If not enough oxygen is being delivered, then the body uses up more of the capillary hemoglobin (increased extraction). This increased extraction results in a decrease in the capillary hemoglobin saturation. This is the feature detected by the present invention. Once the body makes adjustments aimed at increasing the capillary flow through increases in pulse and blood pressure, or through lowering resistance to flow in the arterioles, then the capillary hemoglobin rises close to, but not fully back to, normal values.

It is important to note that the drop in capillary hemoglobin saturation occurs at the very earliest stages of ischemia, when the delivery is only a few percent below normal, and before the tissue is working hard to compensate for the ischemia. In contrast, lactic acidosis is a very late sign, when the tissue has insufficient oxygen and it has exhausted its compensatory means, such that the tissue is now in metabolic failure. Thus, capillary hemoglobin is a much earlier sign of ischemia, and occurs in proportion to the degree of ischemia; while flow and venous oxygenation may miss many local ischemia events, and where pH and lactic acid changes are very late findings and thus unreliable indicators of the degree or presence of ischemia (e.g., false negatives are common).

Ischemia is diagnosed by low local tissue oxygenation, not blood oxygenation or flow. In some cases, arterial blood may be well oxygenated, but the delivery of this arterial blood to the tissue is insufficient (such as with a blood clot); in this case the tissue is indeed ischemic while the arterial blood oxygenation is normal. Blood flow also differs from a direct measure of ischemia. For example, in a cooled patient on heart-lung bypass, blood flow may be very, very low; however, the cooled tissues, whose oxygen need has been reduced by the low temperature, are not ischemic. Similarly, a chronically ischemic heart “hibernates” in order to reduce its own oxygen need, and may not be ischemic at reduced flow. In the above animal study example, flow was controlled sufficiently to allow for a low or zero flow to be consistent with ischemia, but such conclusions cannot be always made so clearly in the living non-experimental subject.

Also, in the above animal study example, power was provided to the device externally. However, as noted earlier, an integrated battery or set of batteries can provide power from within the device, reducing cost of the connection tip. An added advantage of this battery-based approach is that it removes the need for electrical connection to the light source, as an added safety feature.

In this example where the flow to the patient's colon was completely stopped, the signal detected from the tissue was a hemoglobin absorbance signal derived from the capillary bed. While absorbance is ideal for hemoglobin analysis, as described in the preferred embodiment, other interactions may be preferable for other measurements. The interaction with the illuminating light that provides the contrast can include absorbance, polarization, optical rotation, scattering, fluorescence, Raman effects, phosphorescence, or fluorescence decay, and measures of a contrast effect may reasonably include one or more of these effects. Other tissue components could be measured, including NADH, NADPH, cytochromes in their oxidized and reduced forms, or even ischemia or oxygen sensitive dyes. Lastly, when monitoring muscle such as the heart, myoglobin is another protein whose saturation is related to the presence or absence of ischemia. In such cases, a combination of hemoglobin in the capillaries as well as myoglobin in the heart, or just myoglobin in the heart myocytes, can serve as markers of ischemia.

A clinical example of low flow being different than ischemia is in the case of twin-twin anastomosis during a twin pregnancy In some cases, one twin provides venous blood to the other, replacing the arterial supply from the mother's placenta. In such cases, the flow of blood to both fetuses is normal, as can be shown by laser Doppler, an optically-based flow measurement.

During fetal surgery performed on human subjects using a device constructed in accordance with the present invention, ischemia can be detected. In the table below, the “Ischemic” infant is receiving too little arterial blood, while the “Better” infant is receiving too much. A normal infant has a tissue saturation of 25%-30%. Note that the ischemic infant had very low tissue saturations (12%), and ultimately died from this ischemia, nearly a month after a correcting, palliative intervention. Had the infant survived, he likely would have had moderate lung, kidney, and cerebral deficits as well. The test data are shown below in Table 2. In other cases, the arterial flow to one fetus is reduced. Such cases can be detected both the ischemia detection of the present invention as well as by using laser Doppler flow.

TABLE 2
Plot of In Utero Skin Saturation in Better and Ischemic Twins
Before and After In Utero Laser Surgery.

| | |
|---|---|
| Better Twin | Ischemic Twin |

| Patient | Before | After | Before | After |
|---|---|---|---|---|
| 001 | 44% | 32% | 12% | 23% |
| 002 | 23% | 27% | 26% | 32% |
| 003 | 43% | 24% | 32% | 37% |
| 004 | 29% | | 23% | |
| Mean | 35% | 27% | 23% | 31% |
| S.D. | 9% | 4% | 8% | 7% |
| | p = | 0.17 | p = | 0.03 |

As an experimental example of low flow detected by both laser Doppler and the present invention, a pig under anesthesia was placed on heart-lung bypass, which replaces the function of the pig's heart with an adjustable pump. In this case, whole body ischemia can be created simply by slowing the heart pump. A doppler probe was placed on the body's main blood vessel, the aorta, while a sensor constructed in accordance with the present invention was placed on the colon through a surgical incision in the abdomen.

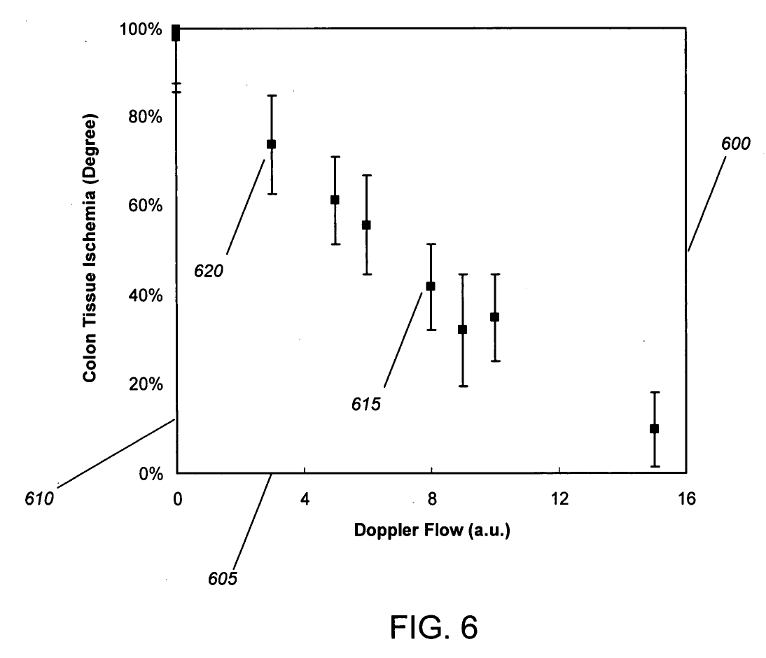

Referring now to FIG. 6, in graph 600, Doppler flow 605 is plotted versus colon tissue ischemia 610. When blood flow is reduced to half of normal, Doppler flow falls from about 14 units to below 8 units, while colon tissue ischemia rises to 44%, as shown by data point 615. When flow is reduced to one-quarter of normal, Doppler flow falls to about 3 units while colon tissue ischemia rises to 75%, as shown as data point 720.

In the above table, the mean difference between the better and ischemic twin was 12% before the surgery, but rose 15% to be only a 3% difference after the surgery. Therefore, this type of feedback can be used to guide the vascular repair using an ischemic signal.

The above description has disclosed an implantable ischemia detector for detecting local tissue ischemia in a quantitative and enabling manner in a broad array of target sites. A working device has been constructed and tested, in which a phosphor-coated white LED and integrated collimating optics have been constructed in accordance with the present invention to produce continuous, broadband light from 400 nm to 700 nm in a collimated beam.

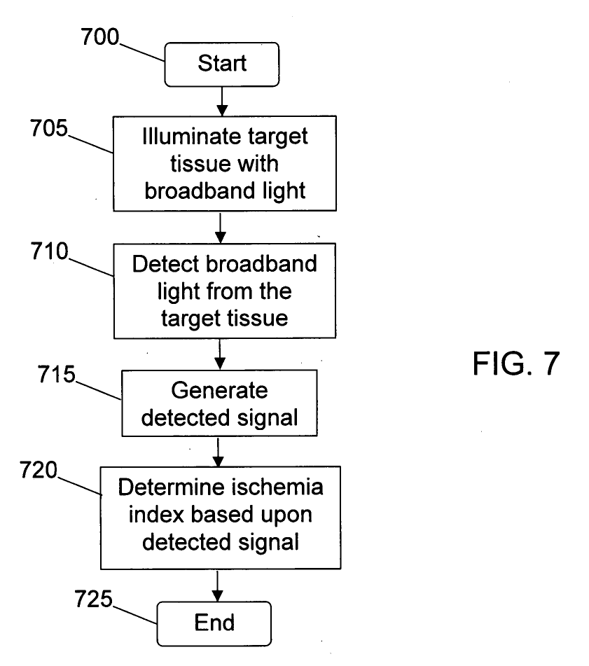

Referring now to FIG. 7, a flow diagram of an exemplary embodiment of local ischemia detection performed by the ischemia detection device constructed in accordance with the present invention is provided. Broadband light is transmitted to a target tissue site in step 705. As described herein above, in accordance with the present invention, the broadband light is characterized to be substantially transmitted through capillaries in the target tissue and substantially absorbed by arterial and venous vessels in the target tissue. Light backscattered by the target site is collected by a sensor in step 710 and a signal representing the detected light is generated in step 715, allowing for an index of ischemia to be determined in step 720. The ischemia index may be subsequently transmitted by a sending unit. Power to the ischemia detection device may be provided by an internal power source, which may in turn be itself powered by an external inductive coil that is brought in proximity to the implanted device in order to provide energy as needed.

As described herein above, the entire implantable device may be encapsulated by a biocompatible shell to add long-term safety while implanted. Used alone, or in combination with an estimate of arterial oxygenation, venous oxygenation, or even of blood flow, this device allows for an index of ischemia to be determined without additional invasiveness beyond the initial implantation. The present device may be interrogated using inductive technology and RF coupling. Implantable devices incorporating the ischemia system, and medical methods of use, have been described. This device has immediate application to several important problems, both medical and industrial, and thus constitutes an important advance in the art.

The foregoing descriptions of specific embodiments and best mode of the present invention have been presented for purposes of illustration and description only. They are not intended to be exhaustive or to limit the invention to the precise forms disclosed. Specific features of the invention are shown in some drawings and not in others, for purposes of convenience only, and any feature may be combined with other features in accordance with the invention. Steps of the described processes may be reordered or combined, and other steps may be included. The embodiments were chosen and described in order to best explain the principles of the invention and its practical application, to thereby enable others skilled in the art to best utilize the invention and various embodiments with various modifications as are suited to the particular use contemplated. Further variations of the invention will be apparent to one skilled in the art in light of this disclosure and such variations are intended to fall within the scope of the appended claims and their equivalents. The publications referenced above are incorporated herein by reference in their entireties.

1. A device for determining local ischemia in a target tissue, the device comprising:
a broadband light source for illuminating the target tissue, wherein the broadband light source is selected such that light is substantially transmitted through capillaries in the target tissue while substantially absorbed through arterial and venous vessels in the target tissue, and for producing a transmitted light substantially representing the capillaries in the target tissue;
a sensor configured to detect light backscattered from the target tissue onto the sensor; and
a ischemia monitoring unit for generating, based on the light detected by the sensor, an output signal that is a function of the presence or degree of local ischemia in the target tissue.

2. The device of claim 1, wherein the broadband light source is a broadband white LED.

3. The device of claim 1, wherein the sensor is a linear CCD configured so as to be wavelength sensitive.

4. The device of claim 1, wherein the ischemia monitoring unit is directly coupled to an implanted pacemaker for the purpose of real-time feedback.

5. The device of claim 3, wherein the output signal is configured to be a function of at least one of tissue hemoglobin or myoglobin saturation.

6. A method of detecting ischemia in a tissue, the method comprising:
implanting a light emitting and detecting device at a target site;
illuminating the target site with locally-sampled visible light illumination from the light emitting and detecting device;
detecting light returning from the site using the light emitting and detecting device; and
determining a measure that is a function of ischemia based upon the detected light.

7. The method of claim 5, further comprising taking interventional medical action based upon the measure.

8. The method of claim 7, wherein taking interventional medical action comprises completing or repositioning a vascular anastomosis during a vascular procedure.

9. The method of claim 7, wherein taking interventional medical action comprises ablating or skipping a contemplated vascular ablation site during a vascular ablation.

10. The method of claim 7, wherein taking interventional medical action comprises accepting or rejecting a vascular repair during a cardiac monitoring procedure.

11. The method of claim 7, wherein taking interventional medical action comprises accepting a surgical repair or performing an additional supply procedure to retain viable tissue with a minimum of ischemic complications during a surgical monitoring procedure.

12. The method of claim 6, wherein taking interventional medical action comprises performing additional vascular surgery during a cardiac vascular procedure based on the measure.

13. An implantable medical device to detect local ischemia in a target tissue, the implantable medical device comprising:
a broadband light source for illuminating the target tissue with light;
a sensor configured so as to detect light backscattered from the target tissue onto the sensor;
a transmitter configured to determine, based upon the light detected by the sensor, an output signal that is a function of the presence or degree of ischemia and to transmit the output signal; and
an exterior shell configured to be biocompatible with respect to implantation.

14. An implantable medical device for detecting local ischemia in a target tissue, the implantable medical device comprising:
a broadband light source for illuminating the target tissue with broadband light;
a sensor configured to detect light backscattered from the target tissue onto the sensor; and
means for determining the presence or absence of local ischemia in the target tissue based on the wavelength of the detected light.

15. A method of detecting ischemia in a target tissue, the method characterized in that a light source selectively emits wavelengths of light to the target tissue such that the wavelengths are substantially transmitted through capillaries in the target tissue and substantially absorbed by arterial and venous vessels in the target tissue.

16. A device for detecting ischemia in tissue, the device comprising a light source configured to selectively emit wavelengths of light to the target tissue such that the wavelengths are substantially transmitted through capillaries in the target tissue and substantially absorbed by arterial and venous vessels in the target tissue.
